(19) United States
(12) Patent Application Publication
(10) Pub. No.: US 2011/0070603 A1
(43) Pub. Date: Mar. 24, 2011

(54) DISC FOR TESTING BLOOD, BLOOD TESTER HAVING THE SAME, AND CONTROL METHOD THEREOF

(75) Inventors: Ji Won KIM, Suwon-si (KR); Beom Seok LEE, Hwaseong-si (KR); In Wook KIM, Seongnam-si (KR); Kui Hyum KIM, Hwaseong-si (KR)

(73) Assignee: SAMSUNG ELECTRONICS CO., LTD., Suwon-si (KR)

(21) Appl. No.: 12/862,138

(22) Filed: Aug. 24, 2010

(30) Foreign Application Priority Data
Sep. 21, 2009 (KR) 10-2009-89100

(51) Int. Cl. C12Q 1/02; C12M 1/34; C12M 1/38

(52) U.S. Cl. 435/29; 435/287.1; 435/287.9; 435/286.1

(57) ABSTRACT

Disclosed are a blood tester for measurement of temperature of a sample placed in a blood-testing disc using a thermochromic pigment, and a control method thereof. The disclosed blood-testing disc, the blood tester having the blood-testing disc loaded therein, and the control method of the same, are used to measure a light absorbance of the thermochromic pigment in order to determine a temperature of a sample when a biological material such as blood, blood serum, blood plasma, or sputum, is subjected to analysis, so that the test may be executed at a constant temperature. Accordingly, more reproducible and accurate results are obtainable, and temperature measurement of a sample may be simply and effectively attained by an optical analyzer built in the blood tester, without an alternative instrument or sensor for measurement thereof.

### CROSS-REFERENCE TO RELATED APPLICATION

This application claims the benefit of Korean Patent Application No. 2009-89100 filed on Sep. 21, 2009 with the Korean Intellectual Property Office, the disclosures of which are incorporated herein by reference.

### BACKGROUND

1. Field

Embodiments relate to a blood tester for measurement of temperature of a sample contained in a blood-testing disc using a thermochromic pigment, and a control method thereof.

2. Description of the Related Art

In general, pathogenic studies require many manual operations and various instruments to analyze biological materials including, for example, blood, blood serum, blood plasma, sputum, etc. In order to conduct an assay by a predetermined process, some stages of the process, e.g., reagent introduction, mixing, separation and migration, reaction, centrifugation, and so forth are executed by manual operation of an experimenter once or several times.

Accordingly, a highly skilled clinical pathologist is required to rapidly implement an assay and such a skilled clinical pathologist also has difficulty in simultaneously conducting several tests. In particular, some assaying methods with different processes such as immunoassay or clinical chemistry require different instruments, leading to more significant difficulties in simultaneous testing. In diagnosis of emergency patients, a rapid examination and results thereof are very important for prompt emergency services. Therefore, an instrument capable of simultaneously, rapidly and correctly conducting a plurality of pathogenic examinations in relation to medical situations is required.

As an example of such instruments, a blood-testing disc including a fluid storage chamber, a channel through which the fluid flows and a valve for regulating the fluid flow, has been developed in order to enable simultaneous, rapid and correct implementation of multiple pathogenic assays, e.g., immunoassay, clinical chemistry, gene study, etc. For instance, after introducing a blood sample into a rotatable blood-testing disc, the disc is rotated, thereby achieving centrifugation to separate serum from the blood sample. The separated serum is then mixed with a desired amount of diluent and transferred to several reaction chambers. Different reagents depending on blood test items are contained beforehand in the chambers and each of such reagents reacts with the serum to generate a specific color. Blood assay is performed by measuring light absorbance based on variation in color.

With regard to the foregoing blood-testing disc, since reagents/samples are generally stored in a chilled state, these are generally kept at room temperature before examination, then used. In this case, a temperature of the sample depends upon a time period during which the sample is maintained at room temperature. In particular, the temperature of the sample is a very important parameter, influencing antigen-antibody reaction, substrate reaction, enzyme reaction, and the like. Therefore, the experimental conditions, including the temperature of a sample at the time of testing is significant for obtaining reproducible results during immunoassay or clinical chemistry.

### SUMMARY

The present disclosure describes a blood tester for measuring the temperature of a sample using a thermochromic pigment, where a material for analysis, such as blood, blood serum, blood plasma, or sputum may be assayed in a blood-testing disc as well as, a method for controlling the blood tester.

Also, a blood tester may control the starting time of a reaction based on when the temperature of a sample reaches a preset level suitable for examination and enables the examination to be executed at a constant temperature whenever the sample is subjected to continuous/discontinuous testing and a control method thereof.

According to an embodiment, a blood-testing disc may include a reaction chamber in which a reagent reacts with a sample, as well as a thermochromic pigment used for measuring a temperature of the sample.

Such a thermochromic pigment may be in any form, including a powder, slurry, master batch or film.

A film type thermochromic pigment may be prepared by applying a powder or liquid thermochromic pigment to a film, which is in turn attached to a blood-testing disc.

A master batch type thermochromic pigment may be prepared by directly adding a pigment to a plastic resin as a raw material for a blood-testing disc.

Such a plastic resin may include polyethylene (PE), polypropylene (PP), polystryrene (PS), polymethyl (meth)acrylate (PMMA), cyclic olefin copolymer (COC), acrylonitrile styrene copolymer (AS) or polyvinyl chloride (PVC).

The temperature range in which the color of a thermochromic pigment may be altered is from −15 to 220° C.

A concentration of the thermochromic pigment may be adjusted depending upon an optical measurement range.

An optically measurable wavelength may range from 200 to 900 nm depending upon the color of the thermochromic pigment.

Examples of a thermochromic pigment include spiropyrans, ethylene compounds (dixanthylene, bianthrone, xanthylideneanthrone), disulfide (diphenyldisulfide, β-dinaphthyldisulfide), or polyamide diacetylene.

The thermochromic pigment may have a main color, including red, rose red, orange, yellow, sky blue, fast blue, dark blue, violet and green, and may also be prepared with other colors by combination of two or more colors.

According to another embodiment a blood tester may include: a blood-testing disc containing a thermochromic pigment; an optical analyzer to measure a light absorbance of the pigment; and a control part that determines the temperature of a sample in the blood-testing disc using the measured light absorbance, determines whether the sample temperature reaches a preset level, and controls the start of assaying the blood-testing disc when the sample temperature reaches the preset level.

The blood-testing disc may include a reaction chamber, in which the reagent reacts with the sample, and a pigment chamber, containing the thermochromic pigment.

The optical analyzer includes: a plurality of light sources to irradiate the pigment chamber with light having different wavelengths; and a plurality of light detection devices to detect a light transmittance of the light penetrating the pigment chamber, wherein the light sources may be aligned to face the corresponding detection devices by interposing the blood-testing disc between the light sources and detection devices.

The plural light sources and the plural detection devices may be arranged opposite to the reaction chamber and the pigment chamber at constant intervals.

The control part may determine the sample temperature by calculating the temperature of the thermochromic pigment according to a signal detected by the light detection device.

An embodiment of the blood tester may further include an identification part to recognize information about the blood-testing disc, while the control part sets a temperature of a sample suitable for immunoassay or clinical chemistry according to the information about the blood-testing disc. The identification part to recognize information may include, but is not limited to, an optical scanning device. The optical device may include a laser scanner capable of reading a bar code.

The control part may determine whether the sample temperature reaches the preset temperature and, if so, may control the start of assaying the blood-testing disc.

When the sample temperature is less than the preset temperature, the control part may control heating of the blood-testing disc, which in turn may enable the sample temperature to reach the preset temperature. The term “preset temperature” as used herein is defined for the purposes of the specification as “a target temperature that is selected by the experimenter and may be between −15 to 220° C.”

According another embodiment a method for controlling a blood tester may include: loading a blood-testing disc containing a thermochromic pigment (into the blood tester); measuring a light absorbance of the thermochromic pigment; measuring a temperature of a sample placed in the blood-testing disc using the measured light absorbance of the thermochromic pigment; and comparing the sample temperature with the preset temperature and, when the sample temperature reaches the preset temperature, starting an assay of the blood-testing disc.

The measurement of light absorbance of the thermochromic pigment may include measuring an degree or amount of variation in color of the thermochromic pigment based on optical transmission.

The measurement of temperature of the sample placed in the blood-testing disc may be performed by calculating a temperature of the thermochromic pigment based on optical transmission and measuring a temperature of the sample according to the calculated temperature of the thermochromic pigment.

The control method may further include: recognizing information about the blood-testing disc; and setting a temperature of a sample suitable for assay of the blood-testing disc based on the information about the blood-testing disc.

The control method may further include heating of the blood-testing disc when the sample temperature is less than the preset temperature.

As described above, according to the disclosed blood-testing disc, the blood tester equipped with the same and the control method thereof, assay of biological substances, such as blood, blood serum, blood plasma, sputum, etc., may start at a constant temperature by measuring a light absorbance of a thermochromic pigment and, in turn, determining a temperature of a sample, so that more reproducible and accurate results are obtainable for test items influenced by temperature as an important parameter. In addition, temperature measurement of a sample may be simply and effectively attained by an optical analyzer built in the blood tester, without an alternative instrument or sensor for measurement thereof. Moreover, an experimenter can select a desired optically measurable wavelength in consideration of use thereof, and also predetermine a temperature range, at which color is varied, in consideration of test purposes. Therefore, the disclosed embodiments may be used in a wide range of applications. The thermochromic pigment used herein may take any form including a powder, slurry, master batch or film, thus being used in a solid state as well as a liquid state in various applications.

### BRIEF DESCRIPTION OF THE DRAWINGS

Hereinafter, exemplary embodiments will be described in greater detail with reference to the accompanying drawings:

FIG. 1 is a view illustrating reaction characteristics of a thermochromic pigment according to an exemplary embodiment;

FIG. 2 is a view illustrating an optical measurement principle of a thermochromic pigment according to an exemplary embodiment;

FIGS. 3A and 3B are graphs illustrating measured results of light absorbance of 1% blue slurry at a wavelength of 630 nm according to an exemplary embodiment;

FIGS. 4A and 4B are graphs illustrating measured results of light absorbance of light blue powders with different concentrations at a wavelength of 630 nm according to an exemplary embodiment;

FIGS. 5A and 5B are graphs illustrating measured results of light absorbance of 0.25% black powder at a wavelength of 450 nm according to another exemplary embodiment;

FIG. 6 is a configuration view illustrating an example of a blood-testing disc containing a thermochromic pigment according to an exemplary embodiment;

FIG. 7 is a configuration view illustrating another example of a blood-testing disc containing a thermochromic pigment according to an exemplary embodiment;

FIG. 8 is a schematic configuration view illustrating a blood tester for measurement of temperature of a sample placed in a blood-testing disc using a thermochromic pigment according to an exemplary embodiment;

FIG. 9 is a schematic configuration view illustrating a blood tester for measurement of temperature of a sample placed in a blood-testing disc using a thermochromic pigment according to another exemplary embodiment;

FIG. 10 is a control block diagram illustrating a blood tester for measurement of temperature of a sample placed in a blood-testing disc using a thermochromic pigment according to an exemplary embodiment; and

FIG. 11 is a flow diagram illustrating a process of controlling measurement of temperature of a sample placed in a blood-testing disc using a thermochromic pigment according to an exemplary embodiment.

### DETAILED DESCRIPTION

Hereinafter, reference will be made in detail to exemplary embodiments without particular restriction to these embodiments.

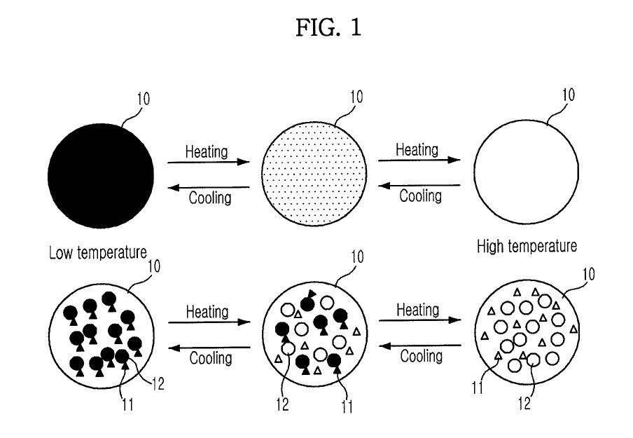

FIG. 1 is a view illustrating reaction characteristics of a thermochromic pigment according to an embodiment of the present disclosure.

Referring to FIG. 1, a thermochromic pigment 10 may be a reversible heat sensitive substance with color variation depending upon temperature, including a microcapsule consisting of an electron donor 11 and an electron acceptor 12.

The thermochromic pigment 10 may generate a color on a crystalline material by interaction between the electron donor 11 and the electron acceptor 12, wherein, when a temperature rises (that is, at a high temperature), the electron acceptor 12 combined with the electron donor 11 is separated therefrom and loses color (becomes transparent). In contrast, when the temperature decreases, the electron acceptor 12 is again combined with the electron donor 11 such that color is generated.

The thermochromic pigment 10 may have a main color including, for example, red, rose red, orange, yellow, sky blue, fast blue, dark blue, violet, green and black and, in addition, may be prepared with other colors by combination of two or more colors.

Examples of materials of the thermochromic pigment 10 may include, for example: spiropyrans; ethylene compounds (e.g., dixanthylene, bianthrone, xanthylideneanthrone); disulfide (e.g., diphenyldisulfide, β-dinaphthyldisulfide); polyamide diacetylene, etc. In addition, the thermochromic pigment may take any form including a powder, slurry, master batch or film. The master batch type pigment may be prepared using a resin selected from PE, PP, PS, PMMA, cyclic olefin copolymer (COC), AS and PVC.

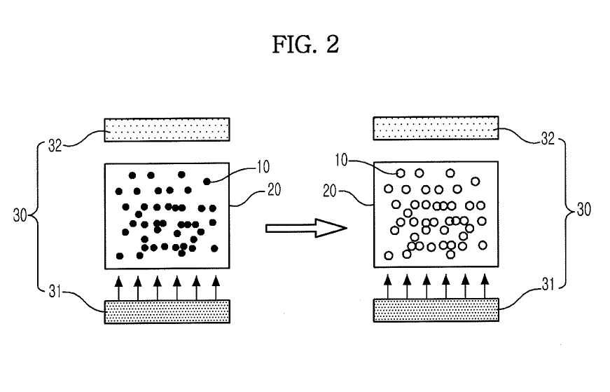

FIG. 2 is a view illustrating an optical measurement principle of a thermochromic pigment according to an exemplary embodiment.

Referring to FIG. 2, an optical analyzer 30 may include a light source 31 (e.g., LED) and a light detection device 32 (e.g., photodiode) that may be disposed by interposing a chamber 20 containing the thermochromic pigment 10 therebetween and may measure variation in color of the thermochromic pigment 10 by applying optical transmission.

Where the light source 31 emits light to the chamber 20 containing the thermochromic pigment 10, optical transmission, that is, light absorbance of the light penetrating the chamber 20, is measured by the light detection device 32. Since the measured light absorbance depends upon variation in color of the thermochromic pigment 10, an internal temperature of the thermochromic pigment 10 may be determined by measuring the light absorbance.

A temperature area in which variation in color of the thermochromic pigment 10 occurs, that is, in which a color of the thermochromic pigment is altered, may range from −15 to 220° C. and the temperature area is optionally defined within this range.

The light source 31 irradiates the chamber 20 containing the thermochromic pigment 10 with light having an optically measurable wavelength in the range of 200 to 900 nm, depending upon color of the thermochromic pigment 10. Accordingly, a concentration of the thermochromic pigment 10 may be adjustable based on the optically measurable wavelength range.

According to color and concentration of the thermochromic pigment 10, an experimenter can select and use a specific light absorbance measured at the optically measurable wavelength, in consideration of test purposes and kinds of samples.

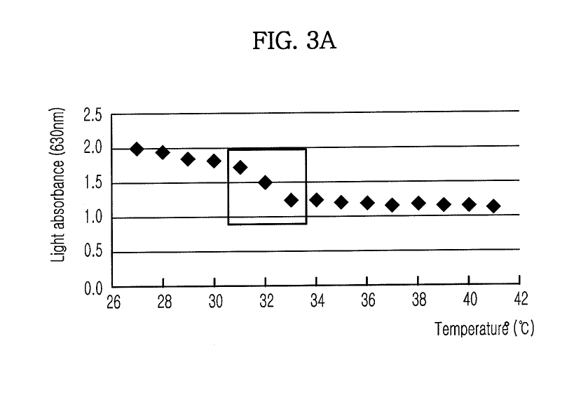

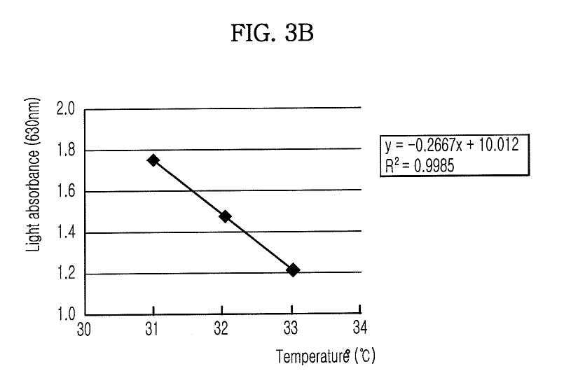

FIGS. 3A and 3B are graphs illustrating measured results of light absorbance of 1% blue slurry at a wavelength of 630 nm according to an exemplary embodiment.

In FIG. 3A, wherein the x-axis is temperature and the y-axis is light absorbance, it was found that a 1% blue slurry comprising the thermochromic pigment 10 becomes transparent in the temperature range of 31 to 33° C., in turn exhibiting reduced light absorbance.

In FIG. 3B, a straight line for light absorbance measured in the temperature range of 31 to 33° C. is expressed by a linear equation of, e.g., y=−0.2667x+10.012.

Here, a correlation coefficient R² for the foregoing thermochromic pigment is 0.9985, which is substantially close to 1, meaning that the light absorbance is closely correlated with the temperature.

Accordingly, for the thermochromic pigment 10 in the form of 1% blue slurry, using the light absorbance measured at 630 nm in the temperature range of 31 to 33° C. may determine a temperature of a sample. Therefore, in consideration of test purposes and kinds of samples, an experimenter can select and use a specific light absorbance.

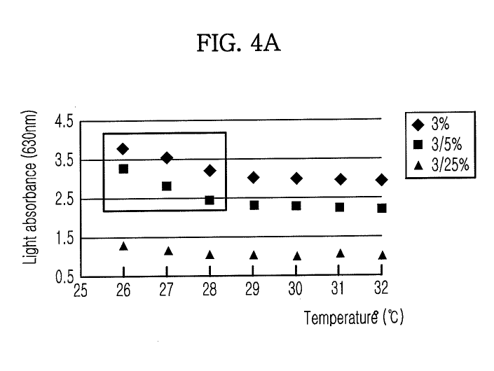

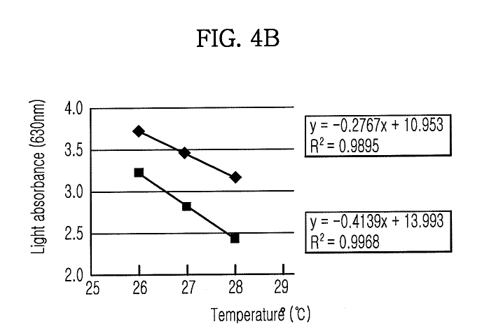

FIGS. 4A and 4B are graphs illustrating measured results of light absorbance of light blue powders with different concentrations at a wavelength of 630 nm according to an exemplary embodiment.

As illustrated in FIG. 3A, when the x-axis is temperature and the y-axis is light absorbance in FIG. 4A, it was found that each of thermochromic pigments 10 in 3% and 3.5% light blue powders, respectively, becomes transparent in the temperature range of 26 to 28° C., in turn exhibiting reduced light absorbance.

In FIG. 4B, a straight line for light absorbance of the 3% light blue powder pigment measured in the temperature range of 26 to 28° C. is expressed by a linear equation of, e.g., y=−0.2767x+10.953, while a straight line for light absorbance of the 3.5% light blue powder pigment measure in the same temperature range is expressed by another linear equation of, e.g., y=−0.4139x+13.993.

Here, a correlation coefficient R² for the 3% light blue powder pigment is 0.9895 and another correlation coefficient R² for the 3.5% light blue powder pigment is 0.9968, each of which is substantially close to 1, meaning that the foregoing pigment exhibits a close correlation between light absorbance and temperature.

Accordingly, for each of thermochromic pigments 10 in the form of 3% blue powder and 3.5% blue powder, respectively, using the light absorbance measured at 630 nm in the temperature range of 26 to 28° C. may determine a temperature of a sample. Therefore, in consideration of test purposes and kinds of samples, an experimenter can select and use a specific light absorbance.

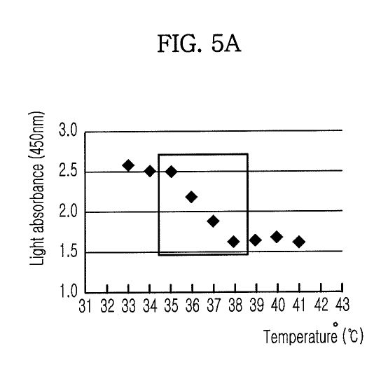

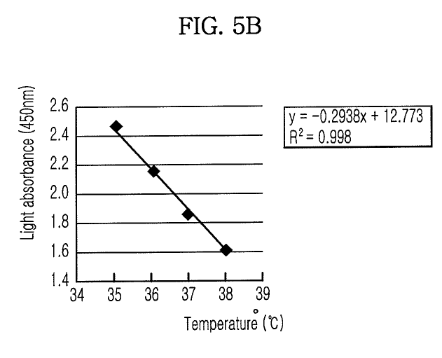

FIGS. 5A and 5B are graphs illustrating measured results of light absorbance of 0.25% black powder at a wavelength of 450 nm according to another embodiment of the present disclosure.

As illustrated in FIGS. 3A and 4A, when the x-axis is temperature and the y-axis is light absorbance in FIG. 5A, it was found that the thermochromic pigment 10 in 0.25% black powder becomes transparent in the temperature range of 35 to 38° C., in turn exhibiting reduced light absorbance.

In FIG. 5B, a straight line for light absorbance measured in the temperature range of 35 to 38° C. is expressed by a linear equation of, e.g., y=−0.2938x+12.773.

Here, a correlation coefficient R² is 0.998, which is substantially close to 1, meaning that the foregoing pigment exhibits a close correlation between light absorbance and temperature.

Accordingly, for the thermochromic pigment 10 in the form of 0.25% black powder, using the light absorbance measured at 450 nm in the temperature range of 35 to 38° C. may determine a temperature of a sample. Therefore, in consideration of test purposes and kinds of samples, an experimenter can select and use a specific light absorbance.

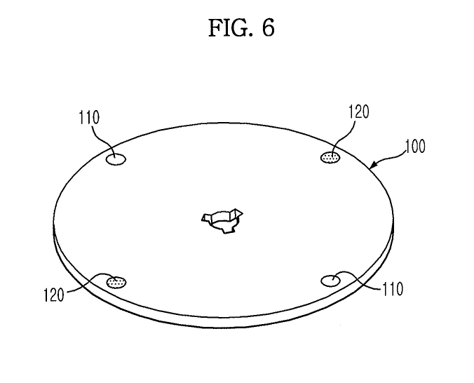

FIG. 6 is a configuration view illustrating an example of a blood-testing disc containing a thermochromic pigment according to an exemplary embodiment.

Referring to FIG. 6, a blood-testing disc 100 includes a reaction chamber 110 in which a reagent reacts with a sample, and a pigment chamber 120, which may contain a thermochromic pigment 10.

More particularly, a rotatable blood-testing disc 100 has two reaction chambers 110 spaced at an interval of 180° angle. A reagent is introduced beforehand into each of the reaction chambers 110, thus reacting with particular ones among ingredients of a sample, in turn generating a color. Depending upon kinds of the reagent, ingredients reacting with the reagent are different and the color is also varied.

Likewise, two pigment chambers 120 are disposed at an interval of 180° angle on a surface of the blood-testing disc 100. Each of the pigment chambers 120 contains the thermochromic pigment 10 in slurry state, wherein the color of the pigment is varied according to temperature range. In particular, as illustrated in FIGS. 3 to 5, the thermochromic pigment 10 contained in the pigment chamber 120 may be prepared with different colors and/or concentrations according to test purposes and kinds of samples.

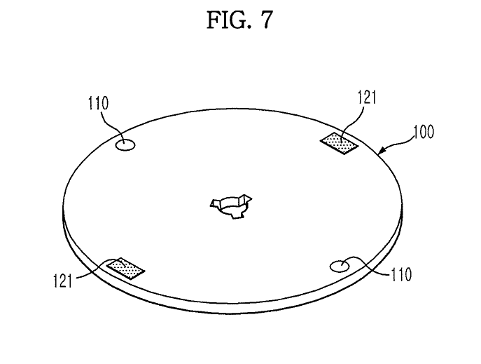

FIG. 7 is a configuration view illustrating another example of a blood-testing disc containing a thermochromic pigment according to an exemplary embodiment.

Referring to FIG. 7, after coating a film 121 with a thermochromic pigment 10 in powder or liquid state, the coated film is fixed and attached to a blood-testing disc 100. Alternatively, a master batch type pigment may be prepared by directly adding a thermochromic pigment 10 to a plastic resin as a raw material of the blood-testing disc 100.

Referring to FIGS. 6 and 7, the blood-testing disc 100 is an illustrative example of a disc used for immunoassay. For a commonly used immunoassay disc, a reaction chamber and a detection chamber may be separately present in the disc. Since the reaction chamber is substantially positioned closer to the back of the disc than the detection chamber, a resultant product may be delivered to the detection chamber through a channel after completing reaction, followed by optical measurement thereof. In this case, the detection chamber does not contain a reagent.

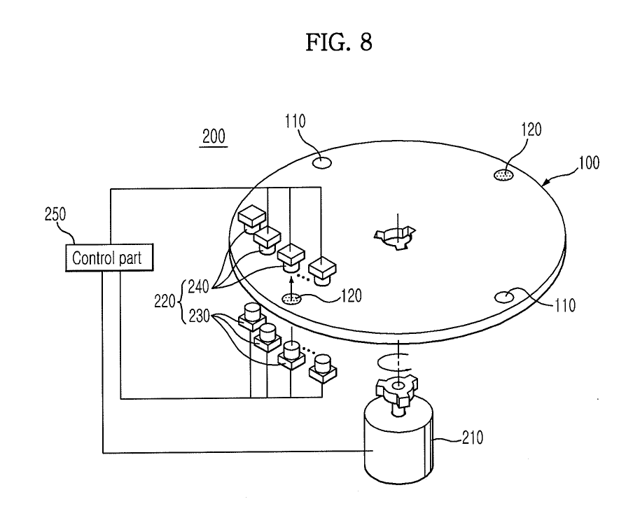

FIG. 8 is a schematic configuration view illustrating a blood tester for measurement of temperature of a sample placed in a blood-testing disc using a thermochromic pigment according to an exemplary embodiment.

Referring to FIG. 8, a blood tester 200 comprises: a centrifugally rotatable blood-testing disc 100 containing a thermochromic pigment 10; a rotating driver 210 to rotate the blood-testing disc 100; an optical analyzer 220 to measure a light absorbance of the thermochromic pigment 10; and a control part 250 that determines a sample temperature using the light absorbance of the thermochromic pigment 10 measured by the optical analyzer 220, and controls the start of assaying the blood-testing disc.

Although a reaction chamber 110 and a pigment chamber 120 only are illustrated in FIG. 8, the blood-testing disc may also have other components in the center thereof, for example: a sample chamber in which a sample such as blood is stored; a dilution chamber in which another reagent such as a diluent miscible with the sample is stored; several channels connecting the foregoing chambers to one another; and/or at least one valve for control of the flow passing through the channels. More detailed description of the blood-testing disc 100 is disclosed in an earlier co-pending application of the present applicant, Korean Laid-Open Patent Application No. 10-2009-0020086, the disclosure of which is incorporated herewith by reference.

If the blood-testing disc 100 with such a configuration is rotated at a high speed, the sample stored in the sample chamber flows toward an outer side of the blood-testing disc 100 via the channel by centrifugal force, so as to be blended with other reagents in turn flowing into the reaction chamber 110.

A shape of the blood-testing disc 100 is not particularly limited to a disc form and may include a sector form disc capable of being fixed to a rotatable frame, in addition to a round type rotating disc. The blood-testing disc 100 is easily formable and may be fabricated using a biologically inactive plastic material such as PMMA, polydimethyl siloxane (PDMS), polycarbonate (PC), etc. Other than the foregoing materials, the blood-testing disc may also be fabricated using any materials with desired chemical and/or biological stability, favorable optical transparency and mechanical workability, without particular restrictions thereto.

Meanwhile, the rotating driver 210 provides centrifugal force to rotate the blood-testing disc 100 at a high velocity, so as to enable the sample to be introduced into the reaction chamber 110 mounted on the blood-testing disc 100. Also, according to rotation of the blood-testing disc 100, the rotating driver 210 guides the reaction chamber 110 and the pigment chamber 120 to face the optical analyzer 220.

The optical analyzer 220 comprises a plurality of, e.g., ten (10) light sources 230 (e.g., LED) which are disposed on a bottom of the blood-testing disc 100 equipped with the reaction chamber 110 and the pigment chamber 120, as well as a plurality of, e.g., 10 light detection devices 240 (e.g., photodiode) which are disposed on a top of the same blood-testing disc 100. An optical measurement device provided in a conventional blood tester is used as the optical analyzer 220.

The plural light sources 230 are aligned opposite to the reaction chamber 110 and the pigment chamber 120 on the blood-testing disc 100 at constant intervals. Likewise, the plural light detection devices 240 are also aligned opposite to the reaction chamber 110 and the pigment chamber 120 on the blood-testing disc 100 at constant intervals. Each of the light sources 230 is arranged to face each corresponding light detection device 240 such that ten (10) different wavelengths are all determined in each of the chambers 110 or 120 during rotation of the blood-testing disc 100.

The plural light sources 230 emit light having an optically measurable wavelength (200 to 900 nm) to the pigment chamber 120 containing the thermochromic pigment 10 depending upon color thereof and the measurable wavelength may be selected by an experimenter in consideration of use thereof. While measuring a light absorbance using one of the light sources 230, the remaining light sources 230 are shut off in order to prevent errors during measurement of the light absorbance.

When light having different wavelengths irradiates the pigment chamber 120 containing the thermochromic pigment 10, optical transmission, e.g., a light absorbance of the light penetrating the pigment chamber 120 is measured in the plural light detection devices 240. Since the light absorbance measured in the plural light detection devices 240 is altered depending upon variation in color of the thermochromic pigment 10, an internal temperature of the thermochromic pigment 10 may be determined by measuring the light absorbance.

The control part 250 functions to control behavior of the rotating driver 210 and the optical analyzer 220. More particularly, the control part detects a rotation phase of the rotating driver 210 and, through rotation phase synchronization, controls measurement of light absorbance of the reaction chamber 110 or the pigment chamber 120 in the optical analyzer 220. For instance, the blood-testing disc 100 has a mark indicating a reference position and controls behavior of the optical analyzer 220 to enable measurement of light absorbance at a site facing the reaction chamber 110 or the pigment chamber 120 using a rotational speed of the disc 100.

The control part 250 measures a light absorbance of the thermochromic pigment 10 penetrating the pigment chamber 12 according to a signal detected by the light detection device 240 of the optical analyzer 220, in turn, calculates an internal temperature of the thermochromic pigment 10 and determines a temperature of a sample using the calculated temperature of the thermochromic pigment 10.

With the measured temperature of the sample according to the light absorbance of the thermochromic pigment 10, the control part 250 determines whether the sample temperature is a preset temperature (e.g., 37° C.) suitable for immunoassay and, if the sample temperature is less than the preset temperature, commands heating of the blood-testing disc 100, and then, controls the start of the immunoassay when the sample temperature reaches the preset temperature. Since the control part 250 has a sample temperature predetermined for immunoassay and stored therein, a sample is taken out from a refrigerator and introduced into the sample chamber built in the blood-testing disc 100, followed by measuring a light absorbance of the thermochromic pigment 10 during rotation of the blood-testing disc 100 in order to execute immunoassay. As a result of measuring the light absorbance and, in turn, determining the sample temperature, the control part controls the start of the immunoassay if the sample temperature reaches a preset level suitable for immunoassay, thereby enabling the start of reaction under a constant temperature condition.

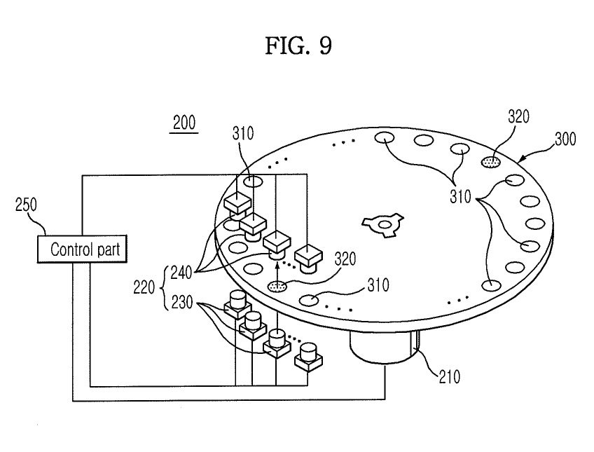

FIG. 9 is a schematic configuration view illustrating a blood tester for measurement of a temperature of a sample placed in a blood-testing disc using a thermochromic pigment, according to another exemplary embodiment. The constitutional components substantially the same as those of FIG. 8 are represented by the same reference numerals and detailed descriptions thereof will be omitted hereinafter to avoid repetition where it may make the subject matter of the disclosure less clear.

Referring to FIG. 9, a blood tester 200 comprises: a rotatable blood-testing disc 300 containing a thermochromic pigment 10; a rotating driver 210 to rotate the blood-testing disc 300; an optical analyzer 220 to measure a light absorbance of the thermochromic pigment 10; and a control part 250 that determines a sample temperature using the light absorbance of the thermochromic pigment 10 measured by the optical analyzer 220, and then, controls the start of assaying the blood-testing disc 300 at a certain temperature.

While two reaction chambers 110 illustrated in FIG. 8 are disposed and spaced from each other at an interval of 180° angle on a top of the rotatable blood-testing disc 100, FIG. 9 shows that a plurality of (i.e., at least 18 units) reaction chambers 310 is arranged at constant intervals on the same radial positions from a top of the blood-testing disc 300. In terms of test items, the reaction chambers contain different type reagents, respectively, provided beforehand therein. Each of such reagents in the reaction chambers 310 reacts with a specific ingredient contained in a sample to generate a color. Reactive materials may depend on kinds of the reagents and may generate different colors.

The blood-testing disc 300 illustrated in FIG. 9 is an example of common discs used for clinical chemistry. Two pigment chambers 310 containing the thermochromic pigment 10 are disposed and spaced from each other at an interval of 180° C. angle on a top of the blood-testing disc 300, which is the same as illustrated in FIG. 8.

Accordingly, with the measured temperature of the sample according to the light absorbance of the thermochromic pigment 10, the control part 250 determines whether the sample temperature is a preset temperature (e.g., 25° C., 30° C., 37° C. . . . ) suitable for clinical chemistry and, if the sample temperature is less than the preset temperature, commands heating of the blood-testing disc 300, and then, controls the start of the clinical chemistry when the sample temperature reaches the preset temperature. Since the control part 250 has a sample temperature predetermined for clinical chemistry and stored therein, a sample is removed from a refrigerator and introduced into the sample chamber built in the blood-testing disc 300, followed by measuring a light absorbance of the thermochromic pigment 10 during rotation of the blood-testing disc 300 in order to execute clinical chemistry. As a result of measuring the light absorbance and, in turn, determining the sample temperature, the control part controls the start of the clinical chemistry if the sample temperature reaches a preset level suitable for clinical chemistry, thereby enabling the start of reaction under a constant temperature condition.

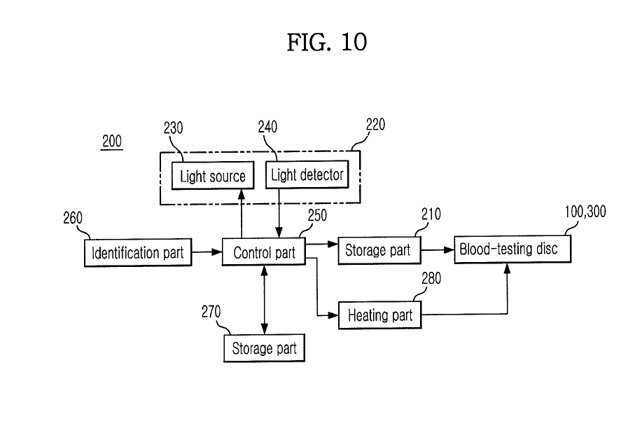

FIG. 10 is a control block diagram illustrating a control process of a blood tester for measurement of temperature of a sample placed in a blood-testing disc using a thermochromic pigment, according to an exemplary embodiment. In this drawing, an identification part 260, a storage part 270 and a heating part 280 are further illustrated in addition of configurations shown in FIGS. 8 and 9.

When the blood-testing disc containing the thermochromic pigment 10 is safely placed on a tray of the blood tester 200, and then, loaded in the blood tester, the identification part 260 recognizes information about the blood-testing disc (e.g., an immunoassay disc or a clinical chemistry disc) and transfers the recognized information to the control part 250.

The storage part 270 provides a database file corresponding to the information about the blood-testing disc 100 or 300 recognized in the identification part 260, to the control part 250. According to the information, the control part 250 controls an optical reaction in the blood-testing disc 100 or 300. For example, after determining whether the blood-testing disc 100 or 300 is an immunoassay disc or a clinical chemistry disc, a specific sample temperature suitable for each of the foregoing testing manners is stored in the control part 250 according to the determined results.

When it is determined whether the sample temperature determined in the control part 250 using the light absorbance of the thermochromic pigment 10 is a preset temperature suitable for immunoassay or clinical chemistry, then, if the sample temperature is less than the preset temperature, the heating part 280 heats the blood-testing disc 100 or 300 according to control commands of the control part 250. The heating part 280 is arranged above and below the tray on which the blood-testing disc 100 or 300 is safely placed, thus heating both sides of the blood-testing disc 100 or 300.

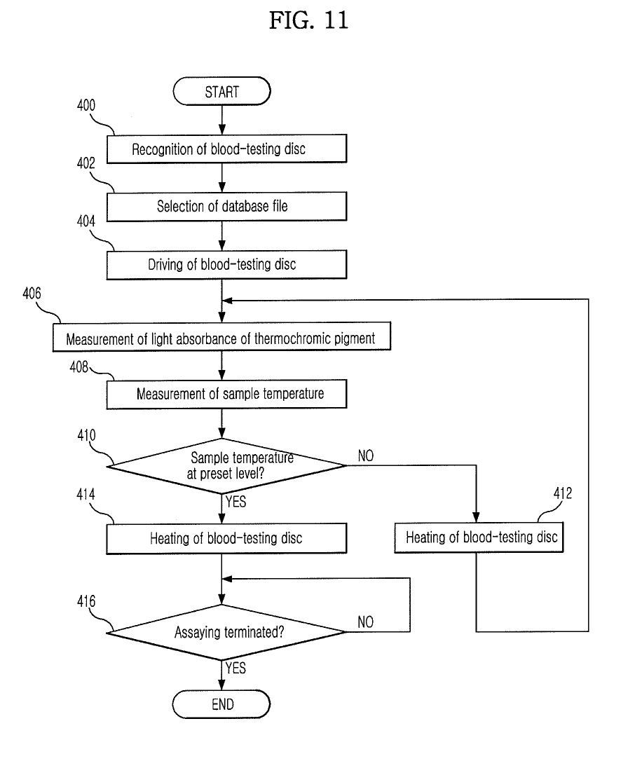

FIG. 11 is a flow diagram illustrating a control process of measuring a sample temperature in a blood tester for measurement of temperature of a sample placed in a blood-testing disc using a thermothromic pigment, according to an exemplary embodiment.

Referring to FIG. 11, in order to execute immunoassay or clinical chemistry, a sample is first removed from a refrigerator and introduced into a sample chamber of the blood-testing disc 100 or 300. After the blood-testing disc 100 or 300 having the sample is safely placed on a tray of the blood tester 200, an identification part 260 recognizes information about the blood-testing disc 100 or 300 (e.g., an immunoassay disc or a clinical chemistry disc) and transfers the recognized information to the control part 250 (400).

Therefore, the control part 250 receives a database file corresponding to the information about the blood-testing disc 100 or 300 recognized in the identification part 260, from the storage part 270, and then, controls an optical reaction in the blood-testing disc 100 or 300 (402).

Afterward, the control part 250 controls a high speed rotation of the blood-testing disc 100 or 300 via the rotating driver 210, based on the database file information (i.e., temperature information of a sample suitable for immunoassay or clinical chemistry) (404).

When the blood-testing disc 100 or 300 is rotated at a high speed, the optical analyzer 220 mounted on a top and a bottom of the blood-testing disc 100 or 300 measures optical transmission, that is, a light absorbance of light penetrating the pigment chamber 120 or 320 containing the thermochromic pigment 100 and transfers the measured light absorbance to the control part 250 (406).

Since the light absorbance measured in the optical analyzer 220 is altered according to variation in color of the thermochromic pigment 10, an internal temperature of the thermochromic pigment 10 may be calculated by measuring the light absorbance.

Before controlling an optical reaction in the blood-testing disc 100 or 300, the control part 250 calculates the internal temperature of the thermochromic pigment 10 using the light absorbance measured by the optical analyzer 220 and a temperature of a sample is determined using the calculated temperature of the thermochromic pigment 100 (408).

As illustrated in FIGS. 3 to 5, the light absorbance of the thermochromic pigment 100 is substantially coincided with a temperature of the same pigment to show a close correlation therebetween. Also, since the temperature of the thermochromic pigment 10 corresponds to a temperature of a sample, the sample temperature may be obtained by measuring the light absorbance of the thermochromic pigment 100.

Accordingly, the control part 250 determines whether the sample temperature measured by the optical analyzer 220 reaches a preset temperature suitable for assaying the blood-testing disc 100 or 300 (410).

As a result of the determination in step 410, if the sample temperature does not reach the preset temperature, the control part 250 controls the heating part 280 to heat both sides of the blood-testing disc 100 or 300 (412), then, returns to step 406 in order to execute further processes.

Alternatively, as a result of the determination in step 410, when the sample temperature reaches the preset temperature, the control part 250 controls the start of immunoassay or clinical chemistry for the blood-testing disc 100 or 300, so as to enable the start of reaction under the same temperature condition (414).

Finally, the control part 250 determines whether the assay of the blood-testing disc 100 or 300 is terminated or not (416) and, if yes, the assay of the blood-testing disc is ended.

Although a few embodiments of the present disclosure have been shown and described, it would be appreciated by those skilled in the art that substitutions, variations and/or modifications may be made in these embodiments without departing from the principles and spirit of the disclosure, the scope of which is defined in the claims and their equivalents.

What is claimed is:

1. A blood-testing disc, comprising:
a reaction chamber in which a reagent contacts a sample; and
a thermochromic pigment measuring a temperature of the sample.

2. The disc according to claim 1, wherein the thermochromic pigment is in a form selected from the group consisting of a powder, slurry, master batch and film.

3. The disc according to claim 2, wherein the thermochromic pigment is in the form of a film that is a film coated with a powder or liquid thermochromic pigment and is attached to the blood-testing disc.

4. The disc according to claim 2, wherein thermochromic pigment is in a form of the master batch in which a pigment is dispersed in a plastic resin, said plastic resin is a raw material for producing the blood-testing disc.

5. The disc according to claim 4, wherein the plastic resin is any one selected from the group consisting of polyethylene, polypropylene, polystryrene, polymethyl (meth)acrylate, cyclic olefin copolymer, acrylonitrile styrene copolymer and polyvinyl chloride.

6. The disc according to claim 1, wherein a temperature range, in which a color of the thermochromic pigment is altered, extends from −15 to 220° C.

7. The disc according to claim 1, wherein a concentration of the thermochromic pigment is adjustable depending upon an optical measurement range.

8. The disc according to claim 7, wherein the optically measurement wavelength ranges from 200 to 900 nm depending upon a color of the thermochromic pigment.

9. The disc according to claim 1, wherein the thermochromic pigment comprises at least one selected from the group consisting of spiropyrans, ethylene compounds, disulfide and polyamide diacetylene.

10. The disc according to claim 1, wherein the thermochromic pigment has any main color selected from the group consisting of red, rose red, orange, yellow, sky blue, fast blue, dark blue, violet and green.

11. A blood tester, comprising:
a blood-testing disc containing a thermochromic pigment;
an optical analyzer to measure a light absorbance of the pigment; and
a control part that determines a sample temperature in the blood-testing disc by measuring light absorbance,
determines whether the sample temperature reaches a preset level, and
controls a start of assaying the blood-testing disc when the sample temperature has reached the preset level.

12. The tester according to claim 11, wherein the blood-testing disc comprises a reaction chamber in which a reagent reacts with the sample and a pigment chamber for containing the thermochromic pigment.

13. The tester according to claim 12, wherein the optical analyzer comprises a plurality of light sources to irradiate the pigment chamber with light having different wavelengths and a plurality of light detection devices to detect a light transmittance of the light penetrating the pigment chamber, wherein the light sources are aligned to face corresponding light detection devices by interposing the blood-testing disc therebetween.

14. The tester according to claim 13, wherein the plural light sources and the plural light detection devices are arranged opposite to the reaction chamber and the pigment chamber at constant intervals.

15. The tester according to claim 13, wherein the control part calculates a temperature of the thermochromic pigment based on a signal detected by the light detection device to determine a temperature of the sample.

16. The tester according to claim 15, further comprising an identification part for recognizing information of the blood-testing disc and wherein the control part sets a temperature of the sample suitable for immunoassay or clinical chemistry.

17. The tester according to claim 16, wherein the control part determines whether the sample temperature reaches the preset temperature and controls the start of assaying the blood-testing disc.

18. The tester according to claim 17, wherein the control part controls heating of the blood-testing disc, thereby enabling the sample temperature to reach the preset temperature, when the sample temperature is less than the preset temperature.

19. A method for controlling a blood tester, comprising:
loading a blood-testing disc containing a thermochromic pigment into a blood tester;
measuring a light absorbance of the thermochromic pigment;
determining a temperature of a sample placed in the blood-testing disc using the measured light absorbance of the thermochromic pigment; and
comparing the sample temperature with a preset level and,
when the sample temperature reaches the preset level, starting the assay in the blood-testing disc.

20. The method according to claim 19, wherein the measurement of light absorbance of the thermochromic pigment comprises applying optical transmission to determine a variation extent in color of the thermochromic pigment.

21. The method according to claim 20, wherein the measurement of temperature of a sample placed in the blood-testing disc is performed by calculating a temperature of the thermochromic pigment based on the optical transmission, and determining the sample temperature according to the calculated temperature of the thermochromic pigment.

22. The method according to claim 19, further comprising:
recognizing information about the blood-testing disc; and
setting a temperature of a sample suitable for assay of the blood-testing disc based on the recognized information.

23. The method according to claim 22, further comprising heating the blood-testing disc when the sample temperature is less than the preset temperature.
